(19) United States
(12) Patent Application Publication
(10) Pub. No.: US 2007/0031467 A1
(43) Pub. Date: Feb. 8, 2007

(54) COMPOSITION AND METHOD FOR VASCULAR EMBOLIZATION

(76) Inventors: John M. Abrahams, Scarsdale, NY (US); Weiliam Chen, Mount Sinai, NY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/425,280

(22) Filed: Jun. 20, 2006

Related U.S. Application Data
(63) Continuation-in-part of application No. 11/447,794, filed on Jun. 6, 2006.
(60) Provisional application No. 60/705,319, filed on Aug. 4, 2005.

(51) Int. Cl. A61F 2/00

(52) U.S. Cl. 424/423

(57) ABSTRACT

A therapeutic composition and a therapeutic method are provided for occlusion of a vascular site. The vascular site may be within a blood vessel or a lymph duct, and may include an aneurysm or an arteriovenous malformation, or may be in a normal section of the vessel or duct. A composition comprises an alkylated chitosan and a polybasic carboxylic acid or an oxidized polysaccharide in an aqueous medium. Another composition comprises a gelatin and an oxidized polysaccharide in an aqueous medium The composition is in the form of a substantially liquid sol immediately upon mixing, and gels or solidifies in situ in the vascular site. The method comprises introducing the composition as a sol into the interior of the vascular site, as with a catheter, wherein it gels in situ. The composition may further include a bioactive agent or a radiopaque agent or both.

### CLAIM OF PRIORITY FROM A PRIOR-FILED PROVISIONAL APPLICATION

This application claims the benefit of priority, under 35 U.S.C. § 119(e), to U.S. Provisional Patent Application No. 60/705,319, filed on Aug. 4, 2005, and, under 35 U.S.C. § 120, to U.S. patent application Ser. No. 11/447,794, filed Jun. 6, 2006, which are incorporated herein by reference. This application is a continuation-in-part, filed under 37 C.F.R. 1.53(b), of U.S. patent application Ser. No. 11/447,794, filed Jun. 6, 2006.

### GOVERNMENT GRANT SUPPORT

This invention was made with the support of the National Institutes of Health under grant no. DK068401 and HL65175. The U.S. Government has certain rights in the invention.

### BACKGROUND

The deliberate embolization of vascular ducts, such as blood vessels or lymph ducts, that is, the deliberate endovascular partial or complete obstruction or occlusion of blood vessels or lymph ducts, is a useful therapeutic process that can be employed in a number of clinical situations. For example, endovascular embolization has been used to control vascular bleeding, to reduce the blood supply to tumors, and to occlude vascular aneurysms, particularly intracranial aneurysms. In recent years, endovascular embolization for the treatment of aneurysms has received much attention.

An aneurysm is a localized dilation of a blood vessel that represents a malcondition with potentially fatal consequences. In an aneurysm, under the pressure exerted by the blood stream, a weakened section of the vessel wall balloons out in excess of the normal diameter of the vessel. Aneurysms can occur in various forms, but all share the feature of a stretched, weakened blood vessel wall. Such a stretched, weakened section of the vessel has an increased probability of rupture, which can result in hemorrhagic stroke if the vessel is within the brain, and can cause potentially life-threatening internal bleeding, especially if the aneurysm is situated in a major artery such as the aorta. Cerebral arteries, such as those making up the circle of Willis, are one of the most common sites for aneurysms, and the rupture of an aneurysm in this location carries a very high risk of severe injury or death from subarachnoid intracerebral hemorrhage.

The wall of a blood vessel is considered to comprise three major layers: the intima (the innermost layer) that is in contact with the blood, formed largely of endothelial cells; the tunica media (middle layer), formed of smooth muscle; and the adventitia (outer layer), formed of connective tissue. A true aneurysm involves the stretching of all three layers. In the development of an aneurysm, an already weakened locus in the blood vessel wall becomes increasingly more vulnerable to further stretching and expansion, leading to an even weaker section of vessel wall. This phenomenon is described by the Laplace Law, which provides that the arterial wall tension is a function of the product of blood pressure and vessel diameter at a given vascular location. As the diameter increases, wall tension increases, possibly resulting in eventual rupture. Also, the aneurysm site is known to breed thrombi, blood clots within the blood stream, that can detach and drift downstream until they encounter a vessel of insufficient diameter, where they can cause a blockage with potentially damaging or fatal consequences.

Endovascular thrombogenic microcoils are gradually becoming the standard of treatment for intracranial aneurysms, including for most posterior circulation and some anterior circulation aneurysms. Although there are numerous variations of the general technology, most are dependent on platinum microcoils of assorted shapes that detach through an electrolytic reaction for deployment in the aneurysm sac. They are typically introduced into the brain vasculature via the femoral artery. Once deployed, microcoils induce arterial stasis within the dome, clot formation and occlusion, and eventual fibrosis with obliteration usually within 12 months. However, despite initial successes, there are pitfalls with this treatment modality. For instance, wide-neck and larger aneurysms are not as effectively treated with traditional endovascular methods, often requiring repeat coiling procedures.¹⁻⁴ Moreover, the most optimal geometry for coiling is when the neck is less than half the size of the dome or when the neck is less than 4 mm.

Previous reports demonstrated that biodegradable polymer (poly-lactide-co-glycolide) coated platinum coil could achieve accelerated fibrosis and obliteration by intensifying aneurismal neointimal formation in animal models.⁵⁻⁶ Other surface modifications include directed cellular responses, ion impingement, and protein coating, aimed to modulate the coil surface properties for complete aneurysm obliteration.⁷⁻²¹ Others have shown that a range of proteins coated onto the coil surface such as albumin, collagen, fibronectin and vascular endothelial growth factor can produce favorable biological responses.^{7-14, 22}

The rate and extent of thrombosis depends on a number of factors including coil composition, packing density, surface charge density, surface texture, and extent of intimal injury.²³ However, coil embolization does not reinforce the weakened blood vessel wall and does not always result in replacement of the aneurysm thrombus with tissue.²⁴ In addition, the long-term consequence of permanently deploying these non-degradable coils into the cerebral vasculature is not known.

The optimal clinical goal of coil embolization in an aneurysm is to induce stasis, thrombosis leading to fibrotic tissue formation, and eventually endothelialization across the aneurysm orifice. However, histopathological evaluation of human aneurysm specimens implanted with platinum microcoils suggested the presence of unorganized clot and fluid spaces between the coils and the aneurysm.²⁵⁻³¹ Even though packing aneurysms with platinum coils appear to increase their stability through thrombosis, due to its relative bio-inertness, platinum contributes little stimulus to fibrotic tissue formation.

Another approach is the direct injection of a liquid polymer embolic agent into the vascular site to be occluded. One type of liquid polymer used in the direct injection technique is a rapidly polymerizing liquid, such as a cyanoacrylate, particularly isobutyl cyanoacrylate, that is delivered to the target site as a liquid, and then is polymerized in situ. Alternatively, a liquid polymer that is precipitated at the target site from a carrier solution has been used. An example of this type of embolic agent is a cellulose acetate polymer mixed with bismuth trioxide and dissolved in dimethyl sulfoxide (DMSO). Another type is ethylene glycol copolymer dissolved in DMSO. On contact with blood, the DMSO diffuses out of the vessel, and the polymer precipitates and rapidly hardens into an embolic mass that can conform to the shape of the aneurysm. Other examples of materials used in this “direct injection” method are disclosed in the following U.S. Pat. Nos. 4,551,132-Pásztor et al.; U.S. Pat. No. 4,795,741-Leshchiner et al.; U.S. Pat. No. 5,525,334-Ito et al.; and U.S. Pat. No. 5,580,568-Greff et al.

Still another approach to the chemical embolization of an abnormal vascular site is the injection into the site of a biocompatible hydrogel, such as poly(2-hydroxyethylmethacrylate) (“pHEMA” or “PHEMA”); or a polyvinyl alcohol foam (“PAF”). See, e.g., Horák et al., “Hydrogels in Endovascular Embolization. II. Clinical Use of Spherical Particles”, Biomaterials, 7, 467 (November, 1986); Rao et al., “Hydrolysed Microspheres from Cross-Linked Polymethyl Methacrylate”, J. Neuroradiol., 18, 61 (1991); Latchaw et al., “Polyvinyl Foam Embolization of Vascular and Neoplastic Lesions of the Head, Neck, and Spine”, Radiology, 131, 669 (June 1979). These materials are delivered as microparticles in a carrier fluid that is injected into the vascular site, a process that has proven difficult to control. Ken (U.S. Pat. No. 6,113,629) has generally disclosed occluding the necks of aneurysms with hydrogels that cross-link and solidify upon exposure to body temperatures. The hydrogel can be used as a carrier for growth factors and a radiopaque agent. However, a continuing need exists for effective, controllable, non-mechanical treatments for aneurysms and other vascular abnormalities requiring repair and/or stabilization.

### SUMMARY

The present invention provides a therapeutic composition and a therapeutic method useful for embolizing, that is, for partially or completely occluding, a endovascular site having a defined interior shape and volume, such as an aneurysm or other arteriovenous malformation. The composition and the method are also useful for embolizing a section of normal blood vessel for the purpose of occluding the vessel, as may be desirable in treatment of a tumor that is vascularized by the blood vessel, or to control downstream bleeding from the blood vessel. The composition and the method can also be used for embolization of other vascular ducts, such as lymph ducts, when such therapy is indicated, such as for repair of a lymphatic leak due to trauma, surgery, or disease.

A composition of the invention comprises a chitosan derivative that has been modified by the introduction of covalently bound moieties onto the polymer chain. The chitosan derivative and a polybasic carboxylic acid or an oxidized polysaccharide, upon dissolution in an aqueous medium, can initially form a flowable, substantially liquid sol that over a period of time, typically in the order of minutes, then gels to form a hydrogel of the invention. The hydrogel, which is biocompatible and can be biodegradable, when formed within a blood vessel or a lymph duct, serves to partially or completely block the flow of fluid through the vessel or duct, resulting in an occlusion, embolization or blockage of the vessel or duct.

An embodiment of the composition of the present invention comprises a flowable aqueous sol of an acrylated chitosan, adjusted to a slightly acidic pH (preferably about 6.0-6.5) such that the acrylated chitosan solution remains a flowable liquid under ambient conditions (i.e., about 20-25° C.) at least for a sufficient period of time for it to be prepared and introduced into a blood vessel or lymph duct. Upon contact with an aqueous medium at near-neutral or slightly alkaline pH such as exists in living human tissue fluids, such as blood or lymph, which have a physiological pH of about 6.9-7.4, the composition of this embodiment solidifies or gels into a hydrogel that totally or partially fills the vascular target site.

Another embodiment of the composition of the invention comprises a flowable aqueous sol including an alkylated chitosan and a polybasic carboxylic acid in an aqueous medium for preparation of a hydrogel for use in vascular occlusion. Another embodiment of the invention comprises an alkylated chitosan, a polybasic carboxylic acid, a carboxylic acid activating reagent or a dehydrating reagent, and an aqueous medium. Within a relatively brief period of time after preparation of a substantially liquid premix sol comprising these components, typically of the order of minutes during which time the premix can be disposed within a vessel or duct of a patient in need thereof, gelation occurs to provide a substantially solid, water-insoluble hydrogel that serves to occlude the vessel or duct.

The invention further provides an embodiment of a flowable aqueous sol comprising a poly(oxyalkylene)chitosan, a hyaluronan, and a carbodiimide for formation of a hydrogel for use in vascular occlusion. In another embodiment, the aqueous solution comprises an acrylated chitosan, a dibasic carboxylic acid, a carbodiimide and a carboxyl activating reagent for formation of a hydrogel for use in vascular occlusion.

Yet another embodiment comprises an acrylated chitosan derivative and an oxidized polysaccharide, such as oxidized dextran or oxidized hyaluronan, for use in vascular occlusion. After formation of the flowable aqueous premix sol, following a period of time during which the sol can be emplaced within a vessel or duct, the sol gels into a hydrogel of the invention that serves to occlude the vessel or duct.

The invention also provides an embodiment of a flowable aqueous sol comprising a gelatin and an oxidized polysaccharide. A specific example is a composition comprising gelatin and oxidized hyaluronan. A mixture of a gelatin and an oxidized hyaluronan in an aqueous medium can be made at a temperature of about 40-45° C., when then gels at a temperature of about 37° C. within about 15-20 minutes.

A composition of the invention can also include a dissolved or dispersed radiopaque agent, for instance, iohexol, allowing the composition to be visualized during and after emplacement using standard angiography techniques.

The invention further provides a method for vascular occlusion. A method of the invention comprises introducing a composition comprising a flowable aqueous premix sol endovascularly so that the premix solidifies or gels in situ to form a hydrogel that can occlude the interior volume of the aneurysm or other arteriovenous malformation, or a section of a normal blood vessel or lymph duct. This flowable aqueous solution may be introduced at the site through a catheter inserted into the vessel or duct.

An embodiment of the method of the invention for vascular occlusion comprises introduction of a flowable aqueous sol including an alkylated chitosan, a polybasic carboxylic acid, and a dehydrating reagent dissolved in an aqueous medium into a section of a blood vessel or lymph duct of a patient in need thereof. Within a relatively brief period of time after introduction of the sol into a vessel or duct, typically in the order of minutes, gelation occurs to provide a substantially solid, water-insoluble hydrogel, which can occlude the vessel or duct.

Another embodiment of the method of the invention further provides for introduction into a blood vessel or lymph duct of a patient in need thereof of a substantially liquid sol comprising an alkylated chitosan, a polybasic carboxylic acid, a carboxylic acid activating reagent or a dehydrating reagent or both, and an aqueous medium, the sol gelling within a period of time to form a substantially solid, water-insoluble hydrogel that serves to occlude the vessel or duct.

Yet another embodiment of the method of the invention comprises introduction of an aqueous sol comprising a mixture of a gelatin and an oxidized hyaluronan in an aqueous medium into a vessel or a duct of a patient in need thereof, wherein the sol gels in situ to form a hydrogel of the invention, which can serve to occlude the vessel or the duct.

The invention further provides therapeutic combinations comprising a composition of the invention and a bioactive agent, the bioactive agent including a plurality of living cells such as regenerative cells, as well as recombinant DNA, cytokines including growth factors such as fibroblast growth factor (FGF) or vascular endothelial growth factor (VEGF), inflammatory agents, anti-inflammatory agents, immunomodulatory agents, or radioactive particles or complexes. Matrix stabilizing agents such as cytochalasin B can also be included. When the agent comprises a polypeptide, heparin or a bioactive fragment or derivative thereof can be mixed with the composition to further stabilize the polypeptide against degradation. The therapeutic combination of the composition and the bioactive agent serves to promote cellular proliferation or regeneration within the volume of the site and to eliminate and heal the abnormal site employing, at least in part, endogenous cellular processes, such as fibrosis, matrix stabilization and the like.

The invention also provides a method for the use of a therapeutic combination of the invention, comprising preparing a substantially liquid sol comprising an embodiment of a premix composition and a bioactive agent, and introducing the sol into a blood vessel or a lymph duct of a patient in need thereof, wherein the sol undergoes gelation in situ to occlude the vessel or duct, so that the bioactive agent is at least partially released with the vessel or the duct.

### BRIEF DESCRIPTION OF THE FIGURES

FIG. 1 Acrylated chitosan (aCHN) gelation in pH 7.4 phosphate buffered saline. Panel (A): initial formation of two phases; (B): gelation of aqueous aCHN phase (circled).

FIG. 2 Schematic illustration of the surgical procedure required for polymer gel infusion. (A) Placement of the permanent distal ligature and temporary proximal ligature on the exposed common carotid artery. (B) Release of the temporary ligature after infusion of polymer gel, another permanent ligature is placed next to the arteriotomy site for closure. There was noticeable dilation of the artery after polymer gel infusion.

FIG. 3 The extent of occlusion of artery two weeks after intervention. (1) Pristine arteries, (2) arteries infused with aCHN polymer gel, (3) arteries infused with saline, (4) arteries infused with VEGF solution, and (5) arteries infused with bioactive VEGF/aCHN polymer gel. The p-values in the figure represent the statistical difference between individual treatment and the arteries receiving VEGF/aCHN polymer gel.

FIG. 4 Representative hematoxylin and eosin stained histological specimens of the Common Carotid Arteries 2 weeks after intervention. (A) VEGF/aCHN polymer gel, (B) aCHN polymer gel only, (C) saline, and (D) VEGF solution.

### DETAILED DESCRIPTION OF PREFERRED EMBODIMENTS

Definitions

As used herein, the term “vascular system” refers to the system of vessels and tissues that carry or circulate fluids such as blood or lymph throughout a living mammalian body. The term “vascular” means of or pertaining to the vascular system. A “vascular site” is a discrete location within the vascular system or a relatively small section of a vascular vessel or duct.

The term “embolize” as used herein refers to obstructing or occluding a volume of a vascular site, either partially or completely, through emplacement of an embolus. When occlusion is complete, fluid flow through the vessel is blocked, whereas partial occlusion allows for diminished fluid flow relative to normal flow past the embolus.

As used herein, a “vascular occlusive composition” refers to a composition of the invention comprising a chitosan derivative. An “effective embolic amount” of a vascular occlusive composition is an amount of the composition sufficient to cause partial or complete occlusion of a vascular vessel or duct.

An “aneurysm” is a localized, blood-filled dilation of a blood vessel.

“Intracranial circulation” means blood circulation within the cranium.

“Posterior circulation” means blood circulation in the posterior cerebral artery.

“Anterior circulation” means blood circulation in the anterior cerebral artery.

“Chitosan,” as the term is used herein, refers to deacetylated chitin, the natural product found in fungi and crustacean shells. Chitosan is polymeric D-glucosamine (2-amino-2-deoxyglucose) linked in the β-1,4 configuration.

An example of a section of a chitosan chain has the following chemical structure, wherein the number of glucosamine units may range from only a few upwards into the hundreds:

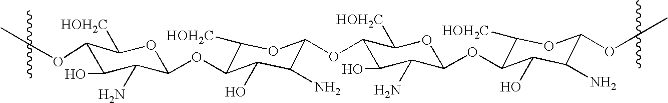

Chitosan is commercially available in a wide range of purities, degrees of polymerization, and degrees of deacetylation, from a number of suppliers. It is biocompatible and biodegradable, and has been used to form films, in biomedical devices and to form microcapsule implants for controlled release in drug delivery. See, e.g., S. Hirano et al., Biochem. Sys. Ecol., 19, 379 (1991); A. D. Sezer, Microencapsulation, 16, 687 (1999); A. Bartkowiak et al., Chem. Mater. 11, 2486 (1999); T. Suzuki et al., Biosci. Bioeng., 88, 194 (1999). Chitosan provides a non-protein matrix for 3-dimensional tissue growth, and activates macrophages for tumoricidal activity. It stimulates cell proliferation and historarchitectural tissue organization. Chitosan is a hemostat, which assists blood clotting and blocks nerve endings reducing pain. Chitosan will gradually depolymerize to release β-D-glucosamine, which initiates fibroblast proliferation, helps in ordered collagen deposition and stimulates increased levels of natural hyaluronic acid synthesis at the vascular trauma site.

When referring to the “molecular weight” of a polymeric species such as an alkylated chitosan, a weight-average molecular weight is being referred to herein, as is well known in the art.

A “degree of substitution” of a polymeric species refers to the ratio of the average number of substituent groups, for example an alkyl substituent, per monomeric unit of the polymer as defined.

A “degree of polymerization” of a polymeric species refers to the number of monomeric units in a given polymer molecule, or the average of such numbers for a set of polymer molecules.

As the term is used herein, an “alkylated chitosan” is a molecular entity formed by reaction of chitosan with carbon-containing molecules. For example, methylation of chitosan, in which bonds are formed between methyl radicals or groups and atoms within the chitosan molecule, such as nitrogen, oxygen or carbon atoms, provides an alkylated chitosan within the definition used herein. Other carbon-containing groups may likewise be chemically bonded to chitosan molecules to produce an alkylated chitosan. For example, poly(oxyalkylene)chitosan and acrylated chitosan are alkylated chitosans within the meaning of the term herein.

A “poly(oxyalkylene)chitosan” is a variety of alkylated chitosan as defined herein. A “poly(oxyalkylene)” group is a polymeric chain of atoms wherein two carbon atoms, an ethylene group, are bonded at either end to oxygen atoms. The carbon atoms of the ethylene group may themselves bear additional radicals. For example, if each ethylene group bears a single methyl group, the resulting poly(oxyalkylene) group is a poly(oxypropylene) group. If the ethylene groups are unsubstituted, the poly(oxyalkylene) group is a poly(oxyethylene) group. A poly(oxyethylene) group may be of a wide range of lengths, or degrees of polymerization, but is of the general molecular formula of the structure [—CH₂—CH₂—O—CH₂—CH₂—O—]ₙ, where n may range from about 3 upwards to 10,000 or more. Commonly but inaccurately referred to as “polyethyleneglycol” or “PEG” derivatives, these polymeric chains are of a hydrophilic, or water-soluble, nature. Thus, a poly(oxyalkylene)chitosan is a chitosan derivative to which poly(oxyalkylene) groups are covalently attached. A terminal carbon atom of the poly(oxyalkylene) group forms a covalent bond with an atom of the chitosan chain, likely a nitrogen atom, although bonds to oxygen or even carbon atoms of the chitosan chain may exist. Poly(oxyethylene)chitosan is often referred to as “polyethyleneglycol-grafted chitosan” or “PEG-g-chitosan.”

The end of the poly(oxyethylene) chain that is not bonded to the chitosan backbone may be a free hydroxyl group, or may comprise a capping group such as methyl. Thus, “polyethylene glycol” or “poly(oxyethylene)” or “poly(oxyalkylene)” as used herein includes polymers of this class wherein one, but not both, of the terminal hydroxyl groups is capped, such as with a methyl group. In a preferred method of preparation of the poly(oxyethylene)chitosan, use of a polyethyleneglycol capped at one end, such as MPEG (methyl polyethyleneglycol) may be advantageous in that if the PEG is first oxidized to provide a terminal aldehyde group, which is then used to alkylate the chitosan via a reductive amination method, blocking of one end of the PEG assures that no difunctional PEG that may crosslink two independent chitosan chains is present in the alkylation reaction. It is preferred to avoid crosslinking in preparation of the poly(oxyethylene)chitosan of the present invention. A representative structure of a poly(oxyethylene)chitosan is shown below.

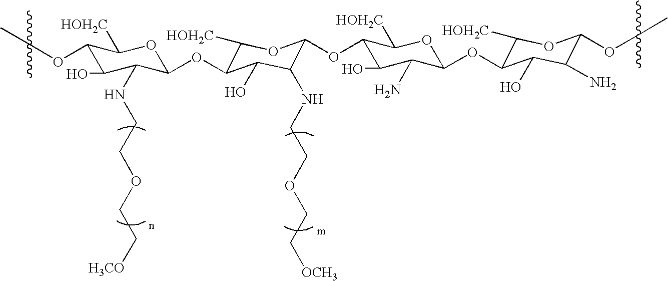

An “acrylated chitosan” as the term is used herein is an alkylated chitosan wherein acrylates have been allowed to react with, and form chemical bonds to, the chitosan molecule. An acrylate is a molecule containing an α,β-unsaturated carbonyl group; thus, acrylic acid is prop-2-enoic acid. An acrylated chitosan is a chitosan wherein a reaction with acrylates has taken place. The acrylate may bond to the chitosan through a Michael addition of the chitosan nitrogen atoms with the acrylate. An example of the chemical structure of a segment of an acrylated chitosan polymer is shown below.

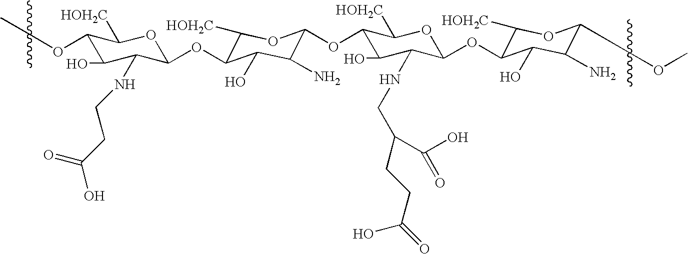

As used herein, a “polybasic carboxylic acid” means a carboxylic acid with more than one ionizable carboxylate residue per molecule. The carboxylic acid may be in an ionized or salt form within the meaning of the term herein. A polybasic carboxylic acid includes a dibasic carboxylic acid within the meaning herein. An alkane dicarboxylic acid is an example, and adipic acid is a more specific example. Disodium adipate is another example. Alternatively, the polybasic carboxylic acid may have hundreds or thousands of ionizable carboxylate groups per molecule; for example, hyaluronan, also known as hyaluronic acid, is also a polybasic carboxylic acid within the meaning assigned herein. The hyaluronan or hyaluronic acid may be in an ionized or salt form, for example sodium hyaluronate, which is a polybasic carboxylic acid within the meaning of the term as used herein.

As used herein, the term “acidic polysaccharide” refers to a polymeric carbohydrate comprising carboxylic acid groups. The polymeric carbohydrate can be naturally occurring, or can be synthetic or semi-synthetic. Examples of acidic polysaccharides are hyaluronan and carboxymethyl cellulose. An oxidized hyaluronan, that is, hyaluronan that has been treated with an oxidizing agent, such as sodium periodate, that cleaves vicinal diol moieties and provides aldehyde groups, is also an acidic polysaccharide within the meaning herein.

As used herein, the term “oxidized polysaccharide” refers to a polymeric carbohydrate that has undergone treatment with an oxidizing reagent, such as sodium periodate, that cleaves vicinal diol moieties of the carbohydrate to yield aldehyde groups. An oxidized hyaluronan, that is, hyaluronan that has been treated with an oxidizing agent, such as sodium periodate, that cleaves vicinal diol moieties and provides aldehyde groups, is an example of an acidic polysaccharide within the meaning herein. An oxidized dextran, that is, dextran that has been treated with an oxidizing agent, such as sodium periodate, that cleaves vicinal diol moieties and provides aldehyde groups, is another example of an oxidized polysaccharide within the meaning herein.

A “dehydrating reagent” as used herein refers to a molecular species that takes up the elements of water from a reaction, serving to drive a coupled reaction due to thermodynamic factors. A dehydrating reagent is an compound that undergoes reaction of covalent bonds upon taking up the elements of water, as opposed to merely absorbing water into physical particles or the like. Preferably a dehydrating reagent is an organic compound. A specific example of a dehydrating reagent is a carbodiimide, that takes up the elements of water and undergoes changes in covalent bonds to ultimately yield a urea derivative.

As used herein, a “carbodiimide” is a class of organic substances comprising a R—N═C═N—R′ moiety. Any organic radicals may comprise the R and R′ groups. A water-soluble carbodiimide is a carbodiimide that has sufficient solubility in water to form a homogeneous solution at concentrations suitable to carry out the gelation reaction as described herein. An example of water-soluble carbodiimide is EDCI, 1-ethyl-3-N,N-dimethylaminopropylcarbodiimide.

A “carboxyl activating reagent” as used herein refers to a molecular species that interacts with a carboxyl group in such a way as to render the carbonyl of the carboxyl group more susceptible to nucleophilic attack, as by an amine to yield an amide. This activation may take place by formation of a complex or by formation of a covalent intermediate. A specific example of a carboxyl activating reagent is an N-hydroxy compound that can form an N-hydroxy ester of the carboxylic acid group, increasing the reactivity of the carbonyl moiety to nucleophilic addition of a molecular species such as an amine. Another example of a carboxyl activating reagent is a carbodiimide. A specific example of a carbodiimide is EDCI.

The term “N-hydroxy compound” refers to an organic compound comprising a chemical bond between a hydroxyl group and a nitrogen atom. Preferred N-hydroxy compounds such as N-hydroxysuccinimide (NHS) and N-hydroxybenztriazole (1-hydroxy benzotriazole) (HBT) are well known in the art as reagents that form esters with carboxylic acid groups and serve to activate the carboxylic acid group in reactions with nucleophiles.

“Gelatin,” as the term is used herein, is a collagen-derived material that is about 98-99% protein by dry weight. The approximate amino acid composition of gelatin is: glycine 21%, proline 12%, hdyroxyproline 12%, glutamate 10%, alanine 9%, arginine 8%, aspartate 6%, lysine 4%, serine 4%, leucine 3%, valine 2%, phenylalanine 2%, threonine 2%, isoleucine 1%, hydroxylysine 1%, methionine and histidine <1% and tyrosine <0.5%.

An “aqueous medium,” as the term is used herein, refers to a medium composed largely, but not necessarily exclusively, of water. Other components may also be present, such as salts, co-solvents, buffers, stabilizers, dispersants, colorants and the like.

The term “iohexol” refers to the compound Iopamidol, N,N′-bis(1,3-dihydroxypropan-2-yl)-5-[[(2S)-2-hydroxypropanoyl]amino]-2,4,6-triiodo-benzene-1,3-dicarboxamide.

A “bioactive agent” as the term is used herein refers to a molecular entity or a cellular entity. As used herein the term thus includes both a chemical or a biochemical substance, referred to as a “molecular entity,” or a plurality of cells, living or dead, in substantially intact biological form, referred to as a “cellular entity.” A molecular entity may be a regenerative agent such as one or more human growth modulating factors such as interleukins, transformation growth factor-b, fibroblast growth factor or vascular endothelial growth factor; or may be a gene therapy agent, a cogener of platelet derived growth factor, a monoclonal antibody directed against growth factors, a drug, or a cell regeneration factor. A cellular entity may be a plurality of drug-producing cells or of regenerative cells such as stem cells. A bioactive agent can be contained within microspheres or nanospheres, which serve to contain and control the release of the agent from the hydrogel.

### DETAILED DESCRIPTION

A novel vascular-occlusive composition of the invention comprises an alkylated chitosan derivative and an aqueous medium. In one embodiment, the alkylated chitosan derivative is an acrylated chitosan. In another embodiment, the alkylated chitosan derivative is a poly(oxyalkylene)chitosan, an example of which is a PEG-chitosan The composition also can comprise a polybasic carboxylic acid. In one embodiment, the polybasic carboxylic acid is an acidic polysaccharide. An example is hyaluronan, also known as hyaluronic acid. Another example is carboxymethylcellulose. In another embodiment, the polybasic carboxylic acid is a linear alkane dicarboxylic acid. A specific example is adipic acid. The composition also can comprise an oxidized polysaccharide. An example is an oxidized dextran.

In an embodiment of a vascular-occlusive composition according to the invention, an alkylated chitosan comprises an acrylated chitosan, which when dissolved in a substantially aqueous medium provides a vascular-occlusive composition. To obtain an acrylated chitosan (“aCHN”) of the invention, chitosan may be reacted with acrylic acid in water solution. The reaction temperature may be in the range of 20-70° C., and the reaction may be allowed to occur for several days, for example about 2-7 days. The acrylated chitosan product may be purified by adjusting the pH of the reaction mixture to alkaline pH, dialyzing against deionized water and lyophilizing to yield N-acrylated chitosan.

In one embodiment of a vascular-occlusive composition, an aCNH can form a flowable solution in an aqueous medium at a pH of less than about 7 and preferably greater than about 6, and gel or solidify at a physiological pH of about 6.9 to about 7.4. This aCHN may comprise a range of degrees of polymerization and degrees of substitution without departing from the principles of the invention, but a preferred degree of substitution of the chitosan backbone with acrylate groups is about 0.25 to about 0.45. A preferred acrylated chitosan has a molecular weight of about 200 kD to about 600 kD. This embodiment of aCHN forms a gel or solid at a physiological pH such that the flowability or liquidity of the composition exhibited at a lower pH ceases. For use as a vascular-occlusive composition, this embodiment of an aCHN is dissolved in an aqueous medium, which may be water or saline, preferably at a pH of about 6.0 to 6.5. A preferred concentration of the aCHN in the aqueous medium is about 1-5% w/v. Additional components such as buffers, preservatives, stabilizers, surfactants, emulsifiers, nutrients, or dispersants may be present in the composition of the invention.

In another embodiment of a composition of the invention, an aCHN forms a hydrogel upon gelation after mixing with a polybasic carboxylic acid and a dehydrating reagent in an aqueous medium. The initial sol gels over a period of time, typically a few minutes, to provide the hydrogel. A subset of polybasic carboxylic acids are linear alkane dicarboxylic acids. A specific example of a linear alkane dicarboxylic acid is adipic acid.

In yet another embodiment of a composition of the invention, an aCHN forms a hydrogel after mixing with an oxidized polysaccharide in an aqueous medium. The initial sol formed after mixing undergoes gelation over a period of time, typically a few minutes, to provide a hydrogel of the invention. A specific example of an oxidized polysaccharide is oxidized dextran.

Another embodiment of an alkylated chitosan comprises a poly(oxyethylene)chitosan. A fully alkylated chitosan monomeric unit has a degree of substitution of 3.0, and a poly(oxyethylene)chitosan according to the present invention may have a degree of substitution ranging up to 3.0 without departing from the principles of the invention. However, a preferred degree of substitution for a poly(oxyethylene)chitosan is about 0.35 to about 0.95. A particularly preferred degree of substitution is about 0.5. It should be understood that other poly(oxyalkylene) groups may be substituted for the poly(oxyethylene) group. For example, a poly(oxypropylene)chitosan may be used in place of, or in addition to, the poly(oxyethylene)chitosan. A preferred poly(oxyethylene)chitosan according to the present invention has a molecular weight of about 200 kD to about 600 kD. A poly(oxyethylene)chitosan of the invention can be prepared by contacting chitosan and a methyl polyethyleneglycol monoaldehyde in the presence of a reducing agent such as sodium cyanoborohydride.

An embodiment of a hydrogel for use in vascular occlusion according to the present invention is a hydrogel that achieves a gelled state from a premix sol. The hydrogel, which may be used to occlude a blood vessel or a lymph duct of a living mammal such as a human patient, is formed upon gelation of the premix, which is in the physical form of a substantially liquid, flowable sol. Mixing of the components that make up a premix provides a liquid or semi-liquid sol that may be pumped or transferred by any technique suitable for handling somewhat viscous liquid materials, such as syringes, pipettes, tubing and the like. Upon standing, the premix sol after a period of time, typically in the order of a few minutes, for example about 1 to about 20 minutes at about 37° C., undergoes gelation to form a hydrogel of the invention.

A premix sol and a resulting hydrogel that forms from the sol are suitable for contact with living biological tissue, being biocompatible and preferably biodegradable. Thus, the hydrogel can remain in contact with living biological tissue within a human patient for an extended period of time without damaging the tissue on which it is disposed. In an embodiment of the invention, the hydrogel serves to occlude fluid flow in a vessel or duct in which the hydrogel is disposed. In an embodiment of the invention, the hydrogel contains therapeutic or protective agents that are released into the surrounding tissues. In an embodiment of the invention, the hydrogel contains microspheres or nanospheres containing therapeutic agents or protective agents that further control the release of the agents from the hydrogel.

In an embodiment of the invention, a premix for a hydrogel for vascular occlusion comprises a hyaluronan. A member of the class of acidic polysaccharides, a hyaluronan bears an ionizable carboxylic acid group on every other monosaccharide residue. The hyaluronan can be in the form of a hyaluronate, that is, with at least most of the carboxylic acid groups being in the ionized or salt form. Sodium hyaluronate is a specific example. The degree of substitution of carboxylic acid groups on the polymer backbone, assuming a monomeric unit comprising the disaccharide formed of one glucuronic acid monosaccharide and one 2-acetamido-2-deoxyglucose monosaccharide, is 1.0. Every monomeric unit (disaccharide unit) bears a single ionizable carboxylic acid group. A hyaluronan may be of any of a wide range of degrees of polymerization (molecular weights), but a preferred hyaluronan has a molecular weight of about 2,000 kD to about 3,000 kD.

Preferably, a premix that includes a poly(oxyalkylene)chitosan also contains a hyaluronan. In one embodiment, the premix comprises a poly(oxyethylene)chitosan, a hyaluronan, and a dehydrating reagent in an aqueous medium. An example of a dehydrating reagent is EDCI. In another embodiment, the premix comprises a poly(oxyethylene)chitosan, a hyaluronan, a dehydrating reagent, and a carboxyl activating reagent in an aqueous medium. An example of a carboxyl activating reagent is NHS.

In another embodiment, a premix that includes an alkylated chitosan also includes a polybasic carboxylic acid comprising a carboxymethylcellulose. A carboxymethylcellulose is a derivative of cellulose (a β-1,4 linked polymer of glucose) wherein hydroxyl groups are substituted with carboxymethyl (—CH₂CO₂H) moieties. It is understood that the term carboxymethylcellulose comprises salts of carboxymethylcellulose, such as the sodium salt. A specific example of a premix comprises acrylated chitosan and carboxymethylcellulose sodium salt. Carboxymethylcellulose, as is well-known in the art, may have varying degrees of substitution. A particularly preferred carboxymethylcellulose according to the present invention has a degree of substitution of about 0.7 and a molecular weight of about 80 kD.

A premix according to the present invention comprises an aqueous medium. An aqueous medium comprises water, and may include other components including salts, buffers, co-solvents, additional cross-linking reagents, emulsifiers, dispersants, electrolytes, or the like.

A premix according to the present invention can comprise a dehydrating reagent. The dehydrating reagent is sufficiently stable when dissolved or dispersed in an aqueous medium to assist in driving the formation of the amide bonds before it is hydrolyzed by water. A type of dehydrating reagent is a carbodiimide, which is transformed to a urea compound through incorporation of the elements of water. A water-soluble carbodiimide, is 1-ethyl-3-(N,N-dimethylpropyl)carbodiimide (EDCI), which is preferred as it is soluble in the aqueous medium and thus does not require a co-solvent or dispersant to distribute it homogeneously throughout the premix. Other water-soluble carbodiimides are also preferred dehydrating reagents.

A premix according to the present invention can comprise a carboxyl activating reagent. A carboxyl activating reagent is a reagent that serves to activate a carboxyl group towards formation of a new bond, such as an amide or ester bond with an amine or a hydroxyl-bearing compound respectively. A carboxyl activating reagent can react with the carboxyl group to form a new compound as an intermediate, which then further reacts with another substance such as an amine to form an amide, or a hydroxyl-bearing compound to form an ester. A preferred carboxyl activating reagent is an N-hydroxy compound. An N-hydroxy compound reacts with a carboxyl group to form an N-hydroxy ester of the carboxylic acid, which can subsequently react with, for example, an amino group to form an amide. A preferred N-hydroxy compound is N-hydroxysuccinimide (NHS). Another preferred N-hydroxy compound is N(1)-hydroxybenzotriazole (HBT).

Another carboxyl activating reagent is a carbodiimide. A carbodiimide reacts with a carboxyl group to form an O-acylisourea, which can subsequently react with, for example, an amine to form an amide, releasing the carbodiimide transformed through covalent addition of the elements of water to a urea compound. A preferred carbodiimide is a water-soluble carbodiimide, for example EDCI.

In an embodiment of the present invention, a carbodiimide may serve both as a dehydrating reagent and as a carboxyl activating reagent. Thus, a premix comprising an alkylated chitosan, a polybasic carboxylic acid, and a carbodiimide is a preferred embodiment according to the present invention. Another embodiment is a premix comprising an alkylated chitosan, a polybasic carboxylic acid, a carbodiimide, and another molecular species wherein that species is a carboxyl activating reagent. Another embodiment is a premix comprising an alkylated chitosan, a polybasic carboxylic acid, a carbodiimide, and another molecular species wherein that species is a dehydrating reagent.

Bioactive agents can be combined with premix solutions. by simply blending commercially available solutions of polypeptides or other agents with the aqueous solutions, with gentle mixing. Cells can likewise be blended with the composition, preferably immediately prior to emplacement to enhance survival of living cells.

A radiopaque material that is optionally incorporated in the composition may be fine particles of a selected radiopaque metal, such as gold, platinum, tantalum or the like. Alternatively, a radiopaque agent can be an iodinated organic compound. A specific example is iohexol.

A bioactive agent can be incorporated into the composition of the invention. The agent can be a molecular entity, such as a regenerative agent such as one or more human growth modulating factors such as interleukins, transformation growth factor-b, fibroblast growth factor (FGF) or vascular endothelial growth factor (VEGF), a gene therapy agent, a cogener of platelet derived growth factor, a monoclonal antibody directed against growth factors; a drug, or a cell regeneration factor. A bioactive agent may also be a cellular entity such as a plurality of drug-producing cells or of regenerative cells such as stem cells.

A hydrogel of the invention can further comprise microspheres or nanospheres, which preferably contain a bioactive agent, the microspheres or nanospheres also controlling the release of the therapeutic agent into the surrounding tissues. A “microsphere” or a “nanosphere” as used herein is a particulate body of dimensions of the order of microns (micrometers) or nanometers respectively, wherein the particulate body may be hollow or solid. Microspheres and nanospheres may be formed of organic or inorganic materials. For example, a nanosphere may comprise a buckminsterfullerene (buckyball), which is organic. Alternatively a nanosphere may comprise microporous glass, which is inorganic. It is understood that the terms encompass solid lipid nanoparticles, wherein the nanosphere particles are formed from a solid lipid. Preferably the microsphere or the nanosphere contains a drug or other substance, the timing of the release of which it is advantageous to control.

Due to the abundance of cationic amino groups in the chitosan structure, it is known that drugs with carboxyl groups can been conjugated thereto and sustained release can be achieved through the hydrolysis of the amide or ester bonds linking drugs to the chitosan molecule. Y. D. Sanzgiri, et al., Pharm. Res., 1, 418 (1990). As a polyelectrolyte, chitosan can also electrostatically conjugate sensitive bioactive agents while preserving their bioactivities and enhancing their stabilities. Such derivatives may be formed with the acrylated chitosan of the present invention, and will likewise serve to provide for sustained release and to preserve the bioactivity and to enhance the stability of the conjugated agent(s).

The abundance of positive charges on the alkylated chitosan enables the electrostatic binding of biologically active proteins such as rhVEGF. This is the most gentle mode of conjugating proteins and thus protecting and preserving the bioactivity of sensitive proteins like rhVEGF. The conjugation of proteins like rhVEGF to the alkylated chitosan also serves as a mechanism for modulating the biological activity of the growth factor, thereby limiting the potential for induction of uncontrolled tissue development.

The types of cells that may be incorporated into the composition include progenitor cells of the same type as those from the vascular site, for example an aneurysm, and progenitor cells that are histologically different from those of the vascular site such as embryogenic or adult stem cells, that can act to stabilize the vasculature and/or to accelerate the healing process. The therapeutic composition comprising cells can be administered in the form of a solution or a suspension of the cells mixed with the polymer solution, such that the cells are substantially immobilized within the vascular site upon gelation of the premix. In the case of a vascular site comprising an aneurysm, this serves to concentrate the effect of the therapeutic agent or the cells within the aneurysm and to provide for release of the agent or of the cells or of cellular products over a course of time.

According to a method of the invention, for instance in treatment of an aneurysm, a catheter can be maneuvered into position in the parent vessel comprising the aneurysm, and the composition of the invention is delivered endovascularly through the catheter into the aneurysm, where the solution solidifies or gels. During introduction of a premix solution comprising a radiopaque material into the aneurysm, the disposition of the solution within the body of the patient can be imaged by common techniques to allow monitoring.

If the composition contains one or more bioactive agents useful to cause healing of an aneurysm, the agent(s) gradually diffuse and disperse from the gel mass into the aneurysm, to promote the growth of a cellular mass (neointima) in the void of the aneurysm. If the composition contains cells, the cells themselves may be either released from the gel or products produced by the cell may be released from the gel.

The method of the present invention can be used to embolize normal or abnormal vascular sites. Abnormal vessel sites that can be treated in addition to cerebral aneurysms include aortic aneuryms, arteriovenous malformations, and other vascular defects such as a fistula (an abnormal duct or passage) or a telangiectasia (chronic dilation of a group of capillaries). Sites on or in normal vasculature can also be treated by embolization of vessels, for example tumors or other abnormal tissue growth can be deprived of their blood supply by vascular emobolization of the vessels supplying the tumor or abnormal tissue growth.

In the case of an aneurysm, a hydrogel of the invention can be used to occlude the entire volume of the aneurysm, as in the case of a fusiform aneurysm or a saccular or berry aneurysm, or the neck of a saccular or berry aneurysm, to reduce the risk of rupture and thrombus formation but allow for continued circulation. In other situations, for example to interrupt the blood supply of a tumor, a more complete blockage of the flow of blood can be achieved. More complete blockage of blood flow may also be employed to prevent downstream hemorrhage, pooling, and other deleterious effects.

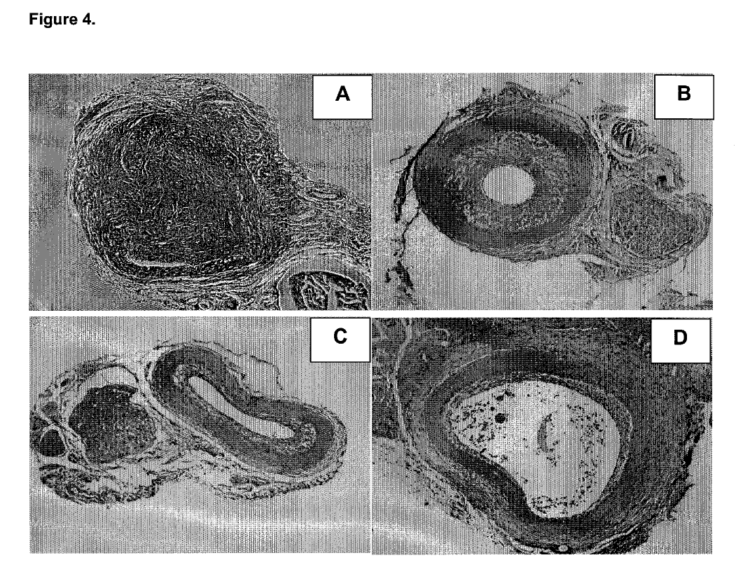

As shown in FIG. 4A, the application of the aCHN-VEGF combination results in a profound response leading to complete filling of the aneursymal sac with fibrous tissue. Interestingly, the application of the aCHN polymer gel alone also resulted in an intense response as indicated by the massive tissue proliferation (FIG. 4B). This effect was likely induced by a combination of inflammatory responses by the presence of aCHN, which induces fibrotic tissue formation, and the stenotic response to arterial injury induced by polymer infusion.

The presence of a stenotic-type response can be substantiated by the moderate tissue proliferation produced by the infusion of saline and VEGF solution (FIGS. 4C & 4D). Nonetheless, the stenotic response alone could not completely account for the profound tissue generation effect of the vessels treated with aCHN alone. Lastly, there was no evidence of angioma development in all the animals treated with rhVEGF. The implication is that the aCHN indeed exerted a certain degree of control on the activity of rhVEGF through electrostatic interaction with its amine groups, thereby, moderating its activity.

The invention will be further described by reference to the following detailed examples wherein both chitosan and acrylic acid were obtained from Sigma-Aldrich (St. Louis, Mo. 63178). The chitosan used was practical grade (>85% deacetylated). The dialysis tubing (MWCO 3,000) was purchased from Spectrum Lab (Racho Dominguez, Calif.). Recombinant human vascular endothelial growth factor (rhVEGF) was obtained from R&D Systems, Minneapolis, Minn. All other chemicals were of reagent grade and distilled and deionized water was used.

### EXAMPLES

### Example 1

Synthesis of Ampholytic Chitosan and Preparation of Bioactive Ampholytic Chitosan Solution

For a typical synthesis, three grams of chitosan was dissolved in 150 ml of 2.75% (v/v) aqueous acrylic acid solution. It was heated and maintained at 50° C. under constant vigorous agitation for 48 hours. Upon cooling to ambient temperature, the pH of the reaction mixture was adjusted to 11 using 1 M NaOH solution. After extensive dialysis for 3 days, the ampholytic chitosan (aCHN) was recovered by lyophilization.

A two percent (w/v) aCHN solution was prepared by dissolving the proper amount of aCHN in water previously adjusted to between pH 6.0 to 6.5. A stock rhVEGF solution (250 ng/μl) was prepared by dissolving rhVEGF in sterile PBS. One hundred microliters of the rhVEGF solution was gently blended with 900 μL of the aCHN solution prepared previously with a micropipette tip to form a bioactive viscous VEGF/aCHN solution.

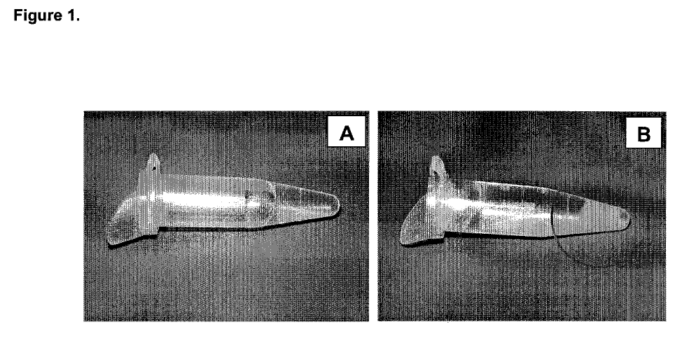

FIG. 1 shows the appearance of an aCHN solution initially (FIG. 1A) and after gelation (FIG. 1B) in the presence of pH 7.4 phosphate buffered saline (PBS). The aCHN solution forms an opaque gel insoluble at physiological pH.

### Example 2

Use of Ampholytic Chitosan to Treat Murine Aneurysm Model

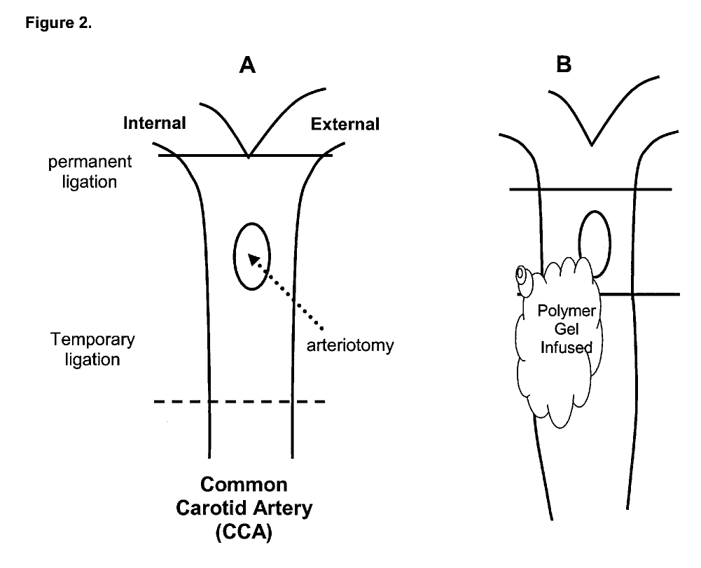

The animal model used was modified from a previously established procedure for adult rats.¹²⁻¹⁷ Sprague-Dawley rats (375 to 450 g) were anesthetized with an intraperitoneal injection of 60 mg/kg sodium pentobarbital and maintained at a temperature of 37° C. throughout the entire procedure. A right paramedian incision was made from the angle of the mandible to the mid-clavicle area. The superficial fascia and muscle layers were separated with blunt dissection until the carotid bundle could be observed. The investing fascia of the common carotid artery (CCA) was incised and the CCA was skeletonized. A permanent ligature was placed proximal to the CCA bifurcation, and a temporary ligature was placed 1 cm distal to the origin of the CCA (FIG. 2). After proximal control of the CCA had been obtained, with complete cessation of arterial blood flow, a small arteriotomy was made 2 mm proximal to the distal ligature. Polymer gel preloaded in a 250 μL Hamilton syringe with a 26-gauge needle was then slowly infused into the CCA. Each animal received a total of 10 μL of the aCHN/VEGF gel (containing a total of 250 ng of VEGF). Likewise, the materials used as controls (aCHN gel, VEGF solution, and saline) were infused into the arteries of the corresponding animals. A new ligature was placed just distal to the arteriotomy, to exclude it from the circulation. The proximal ligature was released to restore blood flow in the CCA segment. Marked vasodilation proximal to the second permanent ligature would occur upon removal of the temporary ligature. The operative field was closed with staples, and the animals were returned to their cages and allowed to recover for two weeks. The animals were administered buphenorphine (0.1-0.5 mg/kg; subcutaneously, daily for 2 days) for pain relief.

Two weeks after the infusion of polymer gel, the rats were euthanized with CO₂. The original incision was reopened and the CCA segment previously infused with polymer gel were resected and preserved in formalin. Following standard histology processing protocols, formalin fixed CCA segments were embedded in paraffin, sectioned, and stained with hematoxylin and eosin. The sections were observed under a microscope (Zeiss Axiovert 200M, Thornwood, N.Y.) and the images were captured and digitized with a camera (AxioCam MRc, Zeiss, Thornwood, N.Y.). The images were analyzed and quantified by the NIH Image J software for their percent occlusion. The data were expressed as mean±standard deviation. Student's t-test was used to determine the statistical differences between groups. Semi-quantitative pathological evaluation on vessel intimal, media and luminal proliferation of the histology sections were performed by a single observer (JMA) who was blinded to the experimental protocol.

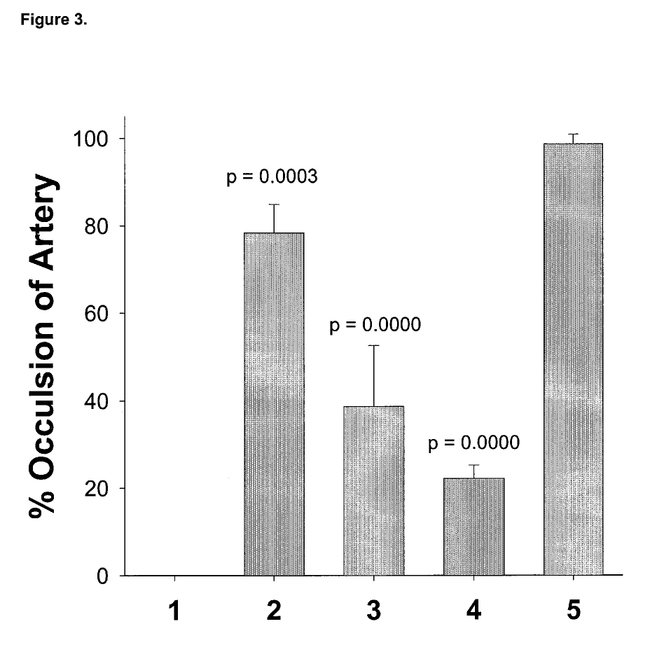

Mean occlusion rates for the vessels are summarized in FIG. 3 with representative histology sections depicted in FIG. 4. As evident from FIG. 4A, the aCHN/VEGF group (n=5) showed virtually complete occlusion of the arterial lumen (98.6±2.2%, FIG. 4A). The aCHN group (n=4) alone showed profound intimal hyperplasia and the lumen was partially filled (78.4±6.5%, FIG. 4B), however, the occlusion was statistically smaller than the aCHN/VEGF group. The saline (n=3) or VEGF solution (n=3) groups showed mild to moderate intimal proliferation response (38.7±13.9% and 22.2±3.1%, respectively, FIGS. 4C and 4D). The control group that received no intervention showed normal appearing vessels (results not shown here). However, there was evidence of vasodilation on gross sectioning of the control vessels.

The results of the pathological scoring for vessel intimal, media and luminal proliferation (all on Grades 0-4) were summarized in Table 1. Comparing scores of the aCHN/VEGF group, results were all significantly greater when compared to other groups (saline, rhVEGF, aCHN). This underscored the advantage of combining the environmentally responsive aCHN gel with VEGF.

TABLE 1
Grading for vessel proliferation. The statistical differences
(p-value) between the group received VEGF-aCHN polymer
gel and other treatments were compared.

| | | Initimal | | Media | | Luminal | |
|---|---|---|---|---|---|---|---|
| | | Pro- | | Pro- | | Pro- | |
| Treatment | N | liferation | p | liferation | p | liferation | p |
| None | 3 | 1.0 ± 0.0 | 0.000 | 1.3 ± 0.6 | 0.000 | 1.0 ± 0.0 | 0.000 |
| Saline | 3 | 2.0 ± 0.0 | 0.000 | 2.3 ± 0.6 | 0.007 | 1.0 ± 0.0 | 0.000 |
| aCHN | 4 | 3.3 ± 0.5 | 0.011 | 3.0 ± 0.0 | 0.010 | 2.3 ± 0.5 | 0.000 |
| polymer | | | | | | | |
| gel | | | | | | | |
| VEGF | 3 | 1.3 ± 0.6 | 0.000 | 1.3 ± 0.6 | 0.000 | 1.0 ± 0.0 | 0.000 |
| solution | | | | | | | |
| VEGF- | 5 | 4.0 ± 0.0 | N/A | 3.8 ± 0.5 | N/A | 4.0 ± 0.0 | N/A |
| aCHN | | | | | | | |
| polymer | | | | | | | |
| gel | | | | | | | |

### Example 3

Preparation of Acrylated Chitosan

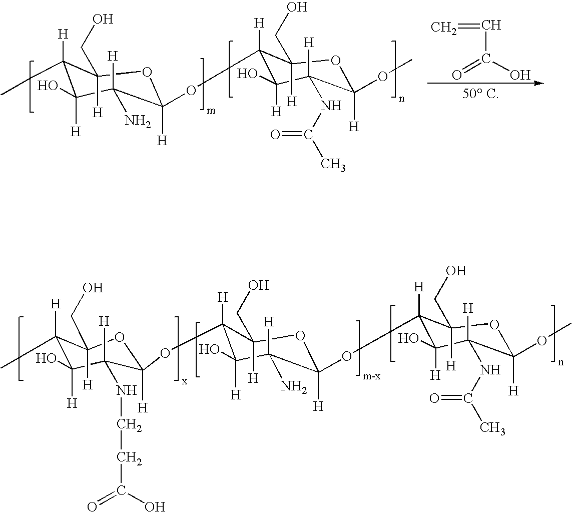

5.52 ml of acrylic acid was dissolved in 150 ml of double distilled water and 3 g of chitosan (Kraeber® 9012-76-4, molecular weight 200-600 kD) was added to it. The mixture was heated to 50C and vigorously stirred for 3 days. After removal of insoluble fragments by centrifugation, the product was collected and its pH was adjusted to 11 by adding NaOH solution. The mixture was dialyzed extensively to remove impurities.

### Example 4

Preparation of PEG-Chitosan

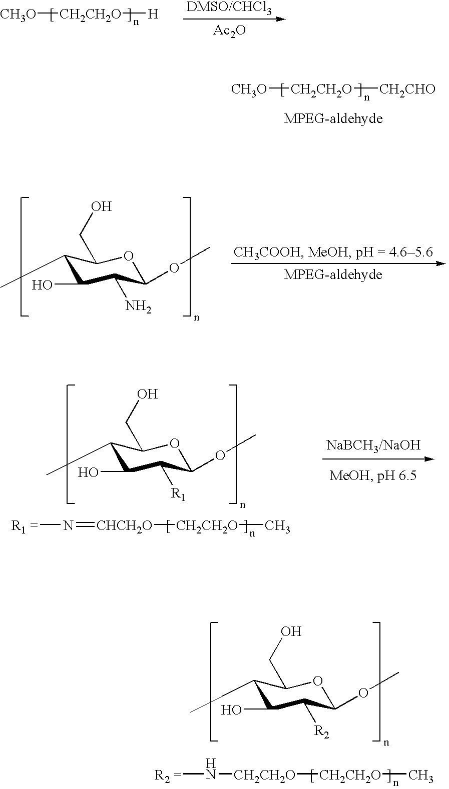

Monomethyl-PEG-aldehyde was prepared by the oxidation of Monomethyl-PEG (MPEG) with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-aldehyde (8 g, DC: 0.40) in aqueous solution was added into the chitosan solution and stirred for 1 h at room temperature. Then the pH of chitosan/MPEG-monoaldehyde solution was adjusted to 6.0-6.5 with aqueous 1 M NaOH solution and stirred for 2 h at room temperature. NaCNBH₃ (0.476 g, 7.6 mmol) in 7 ml water was added to the reaction mixture dropwise and the solution was stirred for 18 h at room temperature. The mixture was dialyzed with dialysis membrane (COMW 6000-8000) against aqueous 0.5 M NaOH solution and water alternately. When the pH of outer solution reached 7.5, the inner solution was centrifuged at 5,000 rpm for 20 min. The precipitate was removed. The supernatant was freeze-dried and washed with 100 ml acetone to get rid of unreacted MPEG. After vacuum drying, the final product (white powder) was obtained as water soluble or organic solvent soluble PEG-g-Chitosan. The yield of water soluble derivatives was around 90% based on the weight of starting chitosan and PEG-aldehyde.

### Example 5

Preparation of a Premix of PEG-chitosan and Hyaluronan

Hyaluronan (sodium hyaluronate, Kraeber 9067-32-7) was dissolved in water as a 0.5% solution by weight. PEG-chitosan, prepared as described in Example 2, was dissolved in water as a 5% solution by weight. A sample of each solution (0.5 mL of each) was mixed, then a solution of EDCI (20 μL of a solution in water at 350 mg/mL) was added and the solution was thoroughly mixed. Immediately a solution of N-hydroxysuccinimide (20 μL of a solution in water at 125 mg/mL) was added and thoroughly mixed in to form a premix. The premix gelled into a hydrogel in about 7 minutes at ambient temperature (22° C.). At 37° C. gelation occurred in about 2 minutes.

### Example 6

Preparation of a Premix of Acrylated Chitosan and Adipic Acid

A sample of acrylated chitosan prepared as described in Example 1 was dissolved in water at a concentration of 2% by weight. A sample of this solution (0.5 mL) was mixed with a solution of adipic acid in water (40 μL of a 20 mg/mL solution), then a solution of EDCI (20 μL of a 350 mg/mL solution) and the solution thoroughly mixed. Then, a solution of N-hydroxysuccinimide in water (20 μL of a 125 mg/mL solution) was mixed in. The premix gelled in about 9 minutes at ambient temperature (22° C.). At 37° C. gelation occurred in about 3 minutes.

### Example 7

Preparation of a Premix of Acrylated Chitosan and Carboxymethylcellulose

A sample of acrylated chitosan prepared as described in Example 1 was dissolved in water at a concentration of 2% by weight. A sample of carboxymethylcellulose sodium salt (Polysciences no. 06140, MW 80 kD, degree of substitution 0.7) was dissolved in water at a concentration of 5% by weight. These two solutions (0.25 mL each) were mixed with a solution of EDCI (20 μL of a 6.5% solution) and the solution thoroughly mixed. Then, a solution of N-hydroxysuccinimide in water (20 μL of a 35% solution) was mixed in. The solution gelled in about 10 minutes at ambient temperature (22° C.).

### Example 8

Preparation of a Premix of Gelatin and Oxidized Dextran

A 20% w/v solution of gelatin in water (1 ml) was mixed with a 20% solution of partially oxidized Hyaluronan (1 ml) (20.3% oxidized). The solution was warmed to about 40-45° C., above the melting point of the gelatin, and was mixed. The solution can be introduced endovascularly at about 37° C., at which temperature gelation occurs in about 15 minutes.

All the citations listed below are incorporated herein by reference.
1. Byrne J V, Sohn M J, Molyneux A J, Chir B. Five-year experience in using coil embolization for ruptured intracranial aneurysms: outcomes and incidence of late rebleeding. J Neurosurg. 1999; 90:656-63.
2. Friedman J A, Nichols D A, Meyer F B, Pichelmann M A, McIver J I, Toussaint L G 3rd, Axley P L, Brown R D Jr: Guglielmi detachable coil treatment of ruptured saccular cerebral aneurysms: retrospective review of a 10-year single-center experience. AJNR Am J Neuroradiol. 2003;24:526-533.
3. Gonzalez N, Murayama Y, Nien Y L, Martin N, Frazee J, Duckwiler G, Jahan R, Gobin Y P, Vinuela F. Treatment of unruptured aneurysms with GDCs: clinical experience with 247 aneurysms. AJNR Am J Neuroradiol. 2004; 25:577-583.
4. Kole M K, Pelz D M, Kalapos P, Lee D H, Gulka I B, Lownie S P. Endovascular coil embolization of intracranial aneurysms: important factors related to rates and outcomes of incomplete occlusion. J Neurosurg 2005; 102:607-615.
5. Murayama Y, Vinuela F, Tateshima S, Gonzalez N R, Song J K, Mahdavieh H, Iruela-Arispe L. Cellular responses of bioabsorbable polymeric material and Guglielmi detachable coil in experimental aneurysms. Stroke 2002;33:1120-1128.
6. Murayama Y, Vinuela F, Tateshima S, Song J K, Gonzalez N R, Wallace M P. Bioabsorbable polymeric material coils for embolization of intracranial aneurysms: a preliminary experimental study. J Neurosurg. 2001; 94:454-463.
7. Murayama Y, Vinuela F, Suzuki Y, Akiba Y, Ulihoa A, Duckwiler G R, Gobin Y P, Vinters H V, Iwaki M, Abe T. Development of the biologically active Guglielmi detachable coil for the treatment of cerebral aneurysms. Part II: an experimental study in a swine aneurysm model. AJNR Am J Neuroradiol. 1999; 20:1992-1999.
8. Kallmes D F, Fujiwara N H, Yuen D, Dai D, Li S T. A collagen-based coil for embolization of saccular aneurysms in a New Zealand White rabbit model. AJNR Am J Neuroradiol 2003; 24:591-596.
9. Matsumoto H, Terada T, Tsuura M, Itakura T, Ogawa A. Basic fibroblast growth factor released from a platinum coil with a polyvinyl alcohol core enhances cellular proliferation and vascular wall thickness: an in vitro and in vivo study. Neurosurgery 2003;53:402-407; discussion 407-408.
10. Murayama Y, Vinuela F, Suzuki Y, Do H M, Massoud T F, Guglielmi G, Ji C, Iwaki M, Kusakabe M, Kamio M, Abe T. Ion implantation and protein coating of detachable coils for endovascular treatment of cerebral aneurysms: concepts and preliminary results in swine models. Neurosurgery 1997; 40:1233-1243; discussion 1243-1244.
11. de Gast A N, Altes T A, Marx W F, Do H M, Helm G A, Kallmes D F. Transforming growth factor beta-coated platinum coils for endovascular treatment of aneurysms: an animal study. Neurosurgery 2001;49:690-694; discussion 694-696.
12. Abrahams J M, Forman M S, Grady M S, Diamond S L. Delivery of human vascular endothelial growth factor with platinum coils enhances wall thickening and coil impregnation in a rat aneurysm model. AJNR Am J Neuroradiol 2001; 22:1410-1417.
13. Marx W E, Cloft H J, Helm G A, Short J G, Do H M, Jensen M E, Kallmes D E. Endovascular treatment of experimental aneurysms by use of biologically modified embolic devices: coil-mediated intraaneurysmal delivery of fibroblast tissue allografts. AJNR Am J Neuroradiol 2001;22:323-33.
14. Murayama Y, Suzuki Y, Vinuela F, Kaibara M, Kurotobi K, Iwaki M, Abe T. Development of a biologically active Guglielmi detachable coil for the treatment of cerebral aneurysms. Part I: in vitro study. AJNR Am J Neuroradiol. 1999;20:1986-1991.
15. Kallmes D F, Williams A D, Cloft H J, Lopes M B, Hankins G R, Helm G A. Platinum coil-mediated implantation of growth factor-secreting endovascular tissue grafts: an in vivo study. Radiology 1998;207:519-523.
16. Abrahams J M, Song C, DeFelice S, Grady M S, Diamond S L, Levy R J. Endovascular microcoil gene delivery using immobilized anti-adenovirus antibody for vector tethering. Stroke 2002;33:1376-1382.
17. Abrahams J M, Forman M S, Grady M S, Diamond S L. Biodegradable polyglycolide endovascular coils promote wall thickening and drug delivery in a rat aneurysm model. Neurosurgery. 2001;49:1187-1193; discussion 1193-1195.
18. Dawson R C, Krisht A F, Barrow D L, Joseph G J, Shengelaia G G, Bonner G. Treatment of experimental aneurysms using collagen-coated microcoils. Neurosurgery. 1995;36:133-139; discussion 139-140.
19. Dawson R C, 3rd, Shengelaia G G, Krisht A F, Bonner G D. Histologic effects of collagen-filled interlocking detachable coils in the ablation of experimental aneurysms in swine. AJNR Am J Neuroradiol. 1996;17:853-858.
20. Kallmes D F, Borland M K, Cloft H J, Altes T A, Dion J E, Jensen M E, Hankins G R, Helm G A. In vitro proliferation and adhesion of basic fibroblast growth factor-producing fibroblasts on platinum coils. Radiology. 1998; 206:237-243.
21. Kwan E S, Heilman C B, Roth P A. Endovascular packing of carotid bifurcation aneurysm with polyester fiber-coated platinum coils in a rabbit model. AJNR Am J Neuroradiol. 1993;14:323-33.
22. Tamatani S, Ozawa T, Minakawa T, Takeuchi S, Koike T, Tanaka R. Radiologic and histopathologic evaluation of canine artery occlusion after collagen-coated platinum microcoil delivery. AJNR Am J Neuroradiol. 1999;20:541-545.
23. Greisler H P. Interactions at the blood/material interface. Ann Vasc Surg. 1990;4:98-103.
24. Byrne J V, Hope J K, Hubbard N, Morris J H. The nature of thrombosis induced by platinum and tungsten coils in saccular aneurysms. AJNR Am J Neuroradiol. 1997; 18:29-33.
25. Bavinzski G, Talazoglu V, Killer M, Richling B, Gruber A, Gross C E, Plenk H. Gross and microscopic histopathological findings in aneurysms of the human brain treated with Guglielmi detachable coils. J Neurosurg. 1999; 91:284-293
26. Castro E, Fortea F, Villoria F, Lacruz C, Ferreras B, Carrillo R. Long-term histopathologic findings in two cerebral aneurysms embolized with Guglielmi detachable coils. AJNR Am J Neuroradiol. 1999; 20:549-552
27. Horowitz M B, Purdy P D, Burns D, Bellotto D. Scanning electron microscopic findings in a basilar tip aneurysm embolized with Guglielmi detachable coils. AJNR Am J Neuroradiol. 1997; 18:688-690
28. Mizoi K, Yoshimoto T, Takahashi A, Nagamine Y. A pitfall in the surgery of a recurrent aneurysm after coil embolization and its histological observation: technical case report. Neurosurgery 1996; 39:165-169
29. Molyneux A J, Ellison D W, Morris J, Byrne J V. Histological findings in giant aneurysms treated with Guglielmi detachable coils. Report of two cases with autopsy correlation. J Neurosurg. 1995; 83:129-132
30. Shimizu S, Kurata A, Takano M, Takagi H, Yamazaki H, Miyasaka Y, Fujii K. Tissue response of a small saccular aneurysm after incomplete occlusion with a Guglielmi detachable coil. AJNR Am J Neuroradiol. 1999; 20:546-548
31. Stiver S I, Porter P J, Willinsky R A, Wallace M C. Acute human histopathology of an intracranial aneurysm treated using Guglielmi detachable coils: case report and review of the literature. Neurosurgery 1998; 43:1203-1208

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills more than one functional requirement as specified by claim language.

All publications, patents and patent applications are incorporated herein by reference. While in the foregoing specification this invention has been described in relation to certain preferred embodiments thereof, and many details have been set for purposes of illustration, it will be apparent to those skilled in the art that the invention is susceptible to additional embodiments and that certain of the details described herein may be varied considerably without departing from the basic principles of the invention.

We claim:

1. A therapeutic composition for embolization of a vascular site comprising an effective embolic amount of an alkylated chitosan and a polybasic carboxylic acid or an oxidized polysaccharide in an aqueous medium.

2. A therapeutic composition for embolization of a vascular site comprising an effective embolic amount of a gelatin and an oxidized polysaccharide, in an aqueous medium.

3. The composition of claim 2 wherein the oxidized polysaccharide comprises oxidized hyaluronan.

4. The composition of claim 1 wherein the alkylated chitosan comprises poly(oxyalkylene)chitosan, the polybasic carboxylic acid comprises hyaluronan, and the composition further comprises a dehydrating reagent and a carboxyl activating reagent.

5. The composition of claim 1 wherein the alkylated chitosan comprises acrylated chitosan and the oxidized polysaccharide comprises oxidized dextran.

6. The composition of claim 1 wherein the alkylated chitosan comprises acrylated chitosan, the polybasic carboxylic acid comprises hyaluronan, and the composition further comprises a dehydrating reagent.

7. The composition of claim 1 wherein the alkylated chitosan comprises acrylated chitosan, the polybasic carboxylic acid comprises an alkane dicarboxylic acid, and the composition further comprises a dehydrating reagent and a carboxyl activating reagent.

8. The composition of claim 1 wherein the alkylated chitosan comprises acrylated chitosan, the polybasic carboxylic acid comprises carboxymethylcellulose, and the composition further comprises a dehydrating reagent and a carboxyl activating reagent.

9. The composition of claim 1 further comprising a dehydrating reagent, a carboxyl activating reagent, or both.

10. The composition of claim 9 wherein the dehydrating reagent is a carbodiimide.

11. The composition of claim 10 wherein the carbodiimide is EDCI.

12. The composition of claim 9 wherein the carboxyl activating reagent is an N-hydroxy compound.

13. The composition of claim 12 wherein the N-hydroxy compound is N-hydroxysuccinimide or N-hydroxybenzotriazole.

14. The composition of claim 9 wherein the carboxyl activating reagent is a carbodiimide.

15. The composition of claim 14 wherein the carbodiimide is EDCI.

16. The composition of claims 1 or 2 further comprising an amount of a bioactive agent effective to stimulate or cause vascular cell growth.

17. The composition of claim 16 wherein the agent is VEGF or FGF.

18. The composition of claim 16 wherein the agent comprises intact cells.

19. The composition of claim 18 wherein the intact cells are progenitor cells of the same type as cells from the vascular site or progenitor cells that are histologically different from cells from the vascular site.

20. The composition of claim 19 wherein the progenitor cells that are histologically different from cells from the vascular site comprise embryogenic or adult stem cells.

21. The composition of claim 16 wherein a bioactive agent is conjugated to the alkylated chitosan either electrostatically or by formation of amide bonds.

22. The composition of claims 1 or 2 wherein the composition in the form of a substantially liquid sol gels into a substantially solid, substantially water-insoluble gel within a period of time of about 1 minute to about 20 minutes at a temperature of about 37° C.

23. The composition of claim 22 wherein the gel is biodegradable within a blood vessel or a lymph duct of a patient.

24. The composition of claims 1 or 2 further comprising a radiopaque material.

25. The composition of claim 24 wherein the radiopaque material comprises iohexol.

26. A method of embolizing a vascular site comprising the interior of a blood vessel or a lymph duct, comprising introducing the composition of claim 1 or claim 2 in the form of a substantially liquid sol into the site so that a hydrogel is formed in situ within the vessel or duct to provide partial or complete occlusion of the vessel or duct.

27. The method of claim 26 further comprising treatment of the vascular site wherein the composition includes a bioactive agent.

28. The method of claim 27 wherein the bioactive agent comprises VEGF or FGF.

29. The method of claim 27 wherein the bioactive agent comprises intact cells that are progenitor cells of the same type as cells from the vascular site or are progenitor cells that are histologically different from cells from the vascular site.

30. The method of claim 29 wherein the progenitor cells that are histologically different from cells from the vascular site comprise embryogenic or adult stem cells.

31. The method of claim 26 wherein the vascular site is a vascular aneurysm.

32. The method of claim 31 wherein the aneurysm is an intracranial aneurysm.

33. The method of claim 32 wherein the intracranial aneurysm is a anterior circulation aneurysm.

34. The method of claim 32 wherein the intracranial aneurysm is a posterior circulation aneurysm.

35. The method of claim 26 wherein the vascular site is disposed in an artery, vein or lymph duct.

36. The method of claim 26 wherein the vascular site is a normal blood vessel or lymph duct, or an aneurysm, a fistula, an arteriovenous malformation, or a telangiectasia.

37. The method of claim 26 wherein the substantially liquid sol is introduced by means of an endovascular catheter.

38. The method of claim 26 wherein the composition comprises no more than about 5 wt-% of the alkylated chitosan.

39. The method of claim 26 wherein the composition further comprises an amount of a bioactive agent effective to stimulate cellular growth in said site.

40. The method of claim 39 wherein the agent is VEGF or FGF.

41. The method of claim 40 wherein the agent VEGF or FGF is stabilized with an effective amount of heparin.

42. The method of claim 26 wherein the composition further comprises a radiopaque material.

43. The method of claim 42 wherein the radiopaque material comprises iohexol.

44. The method of claim 39 wherein the agent comprises intact cells.

45. The method of claim 44 wherein the intact cells are progenitor cells of the same type as cells from the vascular site or progenitor cells that are histologically different from cells from the vascular site.

46. The method of claim 45 wherein the progenitor cells that are histologically different from cells from the vascular site comprise embryogenic or adult stem cells.

47. The method of claim 26 wherein the composition further comprises microspheres or nanospheres or both.

48. The method of claim 47 wherein the microspheres or the nanospheres contain a bioactive agent.

49. The method of claim 48 wherein the microspheres or the nanospheres control the release of the bioactive agent contained therein.
